(19) United States
(12) Patent Application Publication
(10) Pub. No.: US 2025/0321238 A1
(43) Pub. Date: Oct. 16, 2025

(54) MICROFLUIDIC FULLY-AUTOMATIC PLATELET DETECTION METHOD AND PLATELET ANALYSIS AND HOMOGENIZATION SYSTEM

(71) Applicant: Zhejiang Shengyu Medical Technology Co., Ltd, Hangzhou (CN)

(72) Inventors: Lei Zhao, Hangzhou (CN); Hefeng Jin, Hangzhou (CN); Bingfei Yang, Hangzhou (CN); Yu Sheng, Hangzhou (CN); Jiong Sheng, Hangzhou (CN); Shuying Yu, Hangzhou (CN)

(21) Appl. No.: 19/205,564

(22) Filed: May 12, 2025

Related U.S. Application Data
(63) Continuation of application No. PCT/CN2024/087501, filed on Apr. 12, 2024.

(51) Int. Cl. G01N 33/86; B01L 3/00; G01N 21/53; G01N 35/00; G01N 35/10

(52) U.S. Cl. CPC G01N 33/86; B01L 3/50273; B01L 3/502753; G01N 21/534; G01N 35/1002; B01L 2200/025; B01L 2300/047; B01L 2300/0654; B01L 2300/0803; B01L 2400/0409; G01N 2035/00495; G01N 2035/1058

(57) ABSTRACT

The present application relates to a microfluidic fully-automatic platelet detection method and a platelet analysis and homogenization system, where the microfluidic fully-automatic platelet detection method performs a detecting and sampling process of the platelet rich plasma and a detecting and sampling process of the platelet poor plasma on the same platelet analysis and homogenization device, the entire device is small in volume, neither require arrangement of any additional centrifuge device for preparing the platelet poor plasma, nor require arrangement of any additional magnetic stirring device for stirring, the detection steps are simplified, the cost is reduced, and the detection efficiency is improved.

### CROSS REFERENCE TO RELATED APPLICATIONS

This application is a continuation of and incorporates by reference PCT/CN2024/087501 filed on Apr. 12, 2024.

### FIELD OF TECHNOLOGY

The present application relates to the technical field of IVD, and in particular to a microfluidic fully-automatic platelet detection method and a platelet analysis and homogenization system.

### BACKGROUND

LTA (light transmission aggregometry) is the most classic platelet function detection method for determining a platelet aggregation rate. It has low detection costs, relatively good correlation with clinical events, and is relatively popular in clinical applications. Its basic principle is: under specific continuous stirring conditions, an inducer is added into platelet rich plasma (PRP), causing platelets in the PRP to aggregate and the turbidity of the PRP to decrease. A phototube converts a change of the turbidity into an electrical signal and an aggregation curve is plotted on a recorder, from which the platelet aggregation degree can be calculated.

A traditional LTA detection method is to add pre-prepared platelet poor plasma (PPP) into a cuvette and then conduct measurement by projection. After the measurement is completed, the platelet poor plasma (PPP) in the cuvette is pipetted out, and the platelet rich plasma (PRP) and the inducer are added into the cuvette. Detecting is conducted while stirring with a magnetic rod on a magnetic stirring device to obtain detection data.

However, the platelet poor plasma (PPP) needs to be prepared on an additional centrifuge, and it is impossible to complete both detection of the platelet rich plasma (PRP) and detection of the platelet rich plasma (PRP) on the same device while also achieving stirring of the platelet rich plasma (PRP) and the inducer, resulting in complex detection steps, high costs, and low detection efficiency of the traditional LTA detection method.

### SUMMARY

Based on this, it is necessary to provide a microfluidic fully-automatic platelet detection method and a platelet analysis and homogenization system to address the problems of complex steps, high costs and low detection efficiency of using traditional LTA detection methods to detect platelet functions.

In an aspect, the present application provides a microfluidic fully-automatic platelet detection method, including:
acquiring platelet rich plasma and introducing the platelet rich plasma into a disc;
centrifuging the disc to transfer the platelet rich plasma to a detection area of the disc;
introducing an inducing agent into the disc;
centrifuging the disc to transfer the inducing agent to the detection area of the disc;
centrifugally vibrating the disc to allow the platelet rich plasma and the inducing agent to mix and react, and during the centrifugal vibration process, controlling an optical assembly to collect light transmittance data of the detection area;
centrifuging the detection area of the disc to acquire platelet poor plasma; and
centrifuging the disc, and during the centrifugation process, controlling the optical assembly to collect light transmittance data of the detection area.

In another aspect, the present application further provides a microfluidic fully-automatic platelet detection method, including:
acquiring a whole blood sample, and introducing the whole blood sample into a sample addition area of a disc;
centrifuging the disc to separate the whole blood sample into platelet rich plasma and a blood cell sediment;
centrifuging the disc to transfer the platelet rich plasma to a detection area of the disc;
introducing an inducing agent into the disc;
centrifuging the disc to transfer the inducing agent to the detection area of the disc;
centrifugally vibrating the disc to allow the platelet rich plasma and the inducing agent to mix and react, and during the centrifugal vibration process, controlling an optical assembly to collect light transmittance data of the detection area;
centrifuging the detection area of the disc to acquire platelet poor plasma; and
centrifuging the disc, and during the centrifugation process, controlling the optical assembly to collect light transmittance data of the detection area.

In a further aspect, the present application further provides a microfluidic fully-automatic platelet detection method, the method including:
acquiring a whole blood sample, and introducing the whole blood sample into a sample addition area of a disc;
centrifuging the disc to separate the whole blood sample into platelet rich plasma and a blood cell sediment, where the platelet rich plasma moves to a plasma area, and the blood cell sediment moves to a sedimentation area;
centrifuging the disc to transfer the platelet rich plasma from the plasma area to a quantitative area of the disc;
centrifuging the disc to transfer the platelet rich plasma in the quantitative area to a detection area of the disc;
introducing an inducing agent into the disc;
centrifuging the disc to transfer the inducing agent to the detection area of the disc;
centrifugally vibrating the disc to allow the platelet rich plasma and the inducing agent to mix and react, and during the centrifugal vibration process, controlling an optical assembly to collect light transmittance data of the detection area;
centrifuging the detection area of the disc to acquire platelet poor plasma; and
centrifuging the disc, and during the centrifugation process, controlling the optical assembly to collect light transmittance data of the detection area.

In yet a further aspect, the present application further provides a platelet analysis and homogenization system, including:
a disc with a first through hole opened at the center thereof;
a bearing device including a tray, a rotating disc and a supporting component, where the rotating disc is embedded in a groove on a top face of the tray, the tray is provided with at least one light-through hole, a second through hole is opened at the center of the rotating disc, the rotating disc is further provided with a plurality of positioning holes, the disc is fixedly installed on the rotating disc through the positioning holes, and during installation, the first through hole is aligned with the second through hole;
a centrifugal device including a motor and a motor shaft, where the motor shaft passes through the first through hole and the second through hole at the same time, so that when the motor shaft rotates, the rotating disc and the disc are driven to rotate in coordination;
a light source disposed inside the tray and located below the light-through hole;
an optical assembly disposed above the rotating disc and disposed opposite to the light-through hole;
a first control device electrically connected to the motor; and
a second control device electrically connected to the optical assembly.

The present application relates to a microfluidic fully-automatic platelet detection method and a platelet analysis and homogenization system, where the microfluidic fully-automatic platelet detection method performs a detecting and sampling process of the platelet rich plasma and a detecting and sampling process of the platelet poor plasma on the same platelet analysis and homogenization device, the entire device is small in volume, neither requires arrangement of any additional centrifuge device for preparing the platelet poor plasma, nor requires arrangement of any additional magnetic stirring device for stirring, the detection steps are simplified, the cost is reduced, and the detection efficiency is improved.

### BRIEF DESCRIPTION OF THE DRAWINGS

FIG. 1 is a schematic flow chart of a microfluidic fully-automatic platelet detection method provided by an embodiment of the present application.

FIG. 2 is a schematic flow chart of the microfluidic fully-automatic platelet detection method provided by Example 1 of the present application.

FIG. 3 is a schematic flow chart of the microfluidic fully-automatic platelet detection method provided by Example 2 of the present application.

FIG. 4 is a schematic flow chart of the microfluidic fully-automatic platelet detection method provided by Example 3 of the present application.

FIG. 5 is a schematic structural diagram of a single detection unit in a disc A.

FIG. 6 is a schematic structural diagram of a single detection unit in a disc B.

FIG. 7 is a schematic structural diagram of a single detection unit in a disc C.

FIG. 8 is a schematic structural diagram of a single detection unit in a disc A1.

FIG. 9 is a schematic structural diagram of the disc A1.

FIG. 10 is a schematic structural diagram of a single detection unit in a disc A2.

FIG. 11 is a schematic structural diagram of a single detection unit in a disc B1.

FIG. 12 is a schematic structural diagram of a single detection unit in a disc C1.

FIG. 13 is a schematic structural diagram of a platelet analysis and homogenization system provided by an embodiment of the present application (a motor shaft has not been equipped).

FIG. 14 is a schematic structural diagram of a platelet analysis and homogenization system provided by an embodiment of the present application (a motor shaft has been equipped).

### REFERENCE NUMERALS

10-disc; 11-positioning groove; 12-first through hole; 100-detection unit; 110-sample addition area;
120-agent addition area; 130-detection area; 131-barrier area; 132-partition board;
133-detection hole; 140-plasma area; 150-sedimentation area; 160-quantification area; 170-channel;
180-waste liquid pool; 20-bearing device; 210-tray; 211-light-through hole; 220-rotating disc;
221-second through hole; 230-supporting component; 30-centrifugal device; 310-motor; 320-motor shaft;
40-light source; 50-optical assembly; 60-first control device; 70-second control device.

### DESCRIPTION OF THE EMBODIMENTS

In order to make the objectives, technical solutions and advantages of the present application more apparent, the present application is further described in detail hereafter in conjunction with the accompanying drawings and examples. It should be understood that the specific examples described herein are only used for explaining the present application, and are not used for limiting the present application.

The present application provides a microfluidic fully-automatic platelet detection method. Optionally, the microfluidic fully-automatic platelet detection method is applied to a platelet analysis and homogenization system mentioned in the present application.

It should be noted that, the platelet analysis and homogenization system and the platelet analysis and homogenization device mentioned hereafter in the present application are equivalent concepts.

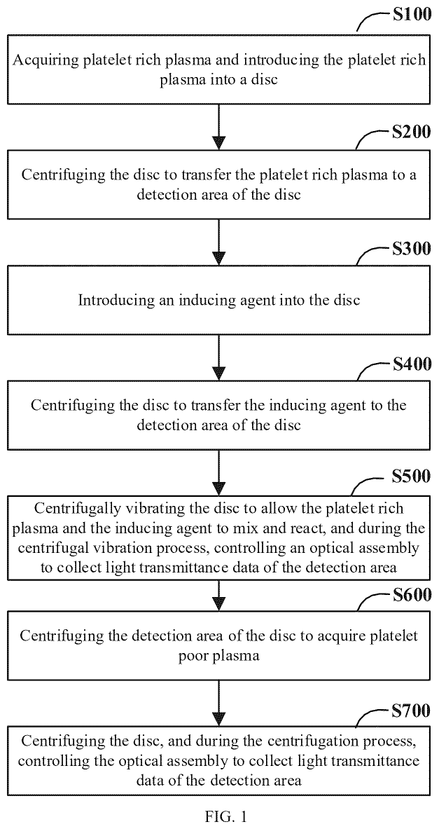

As shown in FIG. 1, in an embodiment of the present application, the method includes the following steps S100 to S700:

S100. platelet rich plasma is acquired and introduced into a disc.

Specifically, the platelet rich plasma is also referred to as PRP (platelet rich plasma).

S200. the disc is centrifuged to transfer the platelet rich plasma to a detection area of the disc.

Specifically, a centrifugal manner may be rotating towards a preset direction.

S300. an inducing agent is introduced into the disc.

Specifically, the inducing agent may be an ADP (adenosine diphosphate) inducing agent. When the inducing agent is introduced into the disc, the inducing agent is introduced into the agent addition area of the disc.

S400. the detection area of the disc is centrifuged to transfer the inducing agent to the detection area of the disc.

Specifically, for the centrifuging, a rotation speed may be 1,000, an acceleration may be 3,000, a deceleration may be 1,000, a centrifugal time may be 5 seconds, and a rotation direction may be clockwise or counterclockwise.

S500. the disc is centrifugally vibrated to allow the platelet rich plasma and the inducing agent to mix and react, and during the centrifugal vibration process, an optical assembly is controlled to collect light transmittance data of the detection area.

Specifically, this step is to perform centrifugal oscillation and detection on a suspension formed by mixing and reacting the platelet rich plasma (PRP) and the inducing agent. The optical assembly may include a light receiving pipe. When the step S500 is conducted, a light source is disposed below the disc, and an optical assembly is disposed above the disc. The light emitted by the light source irradiates the detection area of the disc to generate transmission, and the light transmitted through the detection area is directed toward the light receiving tube in the optical assembly. After the light transmitted through the detection area is collected by the light receiving tube, light transmittance data can be generated and sent to a single-chip microcomputer connected to the optical assembly. The single-chip microcomputer generates a reaction curve based on the light transmittance data, and the reaction curve is used for displaying a detection result of the platelet rich plasma.

S600. the disc is centrifuged to obtain platelet poor plasma.

Specifically, the disc is centrifuged to settle platelet particles in the suspension in the detection area to obtain the platelet poor plasma (PPP), and the relatively clear plasma portion after the settlement of the platelet particles is the platelet poor plasma. During the centrifugation in this step, a rotation speed may be 3,000, an acceleration may be 5,000, a deceleration may be 3,000, a centrifugation time may be 300 seconds, and a rotation direction may be clockwise or counterclockwise.

S700. the disc is centrifuged, and during the centrifugation process, the optical assembly is controlled to collect light transmittance data of the detection area.

Specifically, this step is to perform centrifugation and detection on the platelet poor plasma obtained in the step S600. During centrifugation, a rotation speed may be 120, a centrifugal time may be 10 seconds, and a rotation direction may be clockwise or counterclockwise. A detecting and sampling process of the platelet rich plasma and a detecting and sampling process of the platelet poor plasma are performed on the same platelet analysis and homogenization device. There is neither a need of arrangement of any additional cuvette, any magnetic stirring device and magnetic rod for mixing uniformly, nor a need for any additional centrifuge to prepare the platelet poor plasma. Thus, the cost is greatly reduced, and the operations are carried out on the same device, which simplifies the operation for a staff.

In this embodiment, the detecting and sampling process of the platelet rich plasma and the detecting and sampling process of the platelet poor plasma are performed on the same platelet analysis and homogenization device. The entire device is small in volume, neither requires arrangement of any additional centrifuge device for preparing the platelet poor plasma, nor requires arrangement of any additional magnetic for stirring, the detection steps are simplified, the cost is reduced, and the detection efficiency is improved.

In an embodiment of the present application, the step S100 includes the following steps:

S110. platelet rich plasma is acquired, and the platelet rich plasma is introduced into a sample addition area of the disc.

Specifically, the sample addition area may be a sample addition hole. In this embodiment, the platelet rich plasma can be prepared by an external device and then added into the sample addition area.

A manner for introducing the platelet rich plasma into the sample addition area of the disc may be adding a sample by using a sample injection needle. The sample injection needle can complete the sample addition by injecting the sample once, i.e., single injection. A single sample addition volume may be within a numerical range of greater than or equal to 80 μL and less than or equal to 120 μL. Optionally, the single sample addition volume may be 108 L.

In this embodiment, after the performance of the step S110 is completed, the disc is centrifuged when the step S200 is performed, which is to transfer the platelet rich plasma directly from the sample addition area to the detection area of the disc. Under this transfer manner, during centrifugation a rotation speed may be 1,000, an acceleration may be 3,000, a deceleration may be 3,000, a centrifugal time may be 5 seconds, and a rotation direction may be clockwise or counterclockwise.

In an embodiment of the present application, the step S300 includes:

S310. an inducing agent is acquired and introduced into an agent addition area of the disc.

Specifically, the agent addition area may be an agent addition hole. In this embodiment, the inducing agent can be prepared by an external device and then added into the agent addition area.

In an embodiment of the present application, the step S100 includes the following steps S121 to S122:

S121. a whole blood sample is acquired, and introduced into a sample addition area of a disc.

S122. the disc is centrifuged to separate the whole blood sample into platelet rich plasma and a blood cell sediment.

Specifically, in this embodiment, the preparation of the platelet rich plasma is completed on the disc, that is, the preparation is completed on the platelet analysis and homogenization system, rather than outside the system. By the once disc centrifugation of the step S122, the whole blood sample is separated into the platelet rich plasma and the blood cell sediment, thus completing the preparation of the platelet rich plasma on the disc.

In this embodiment, by introducing the whole blood sample into the addition area of the disc and centrifuging the disc to separate the whole blood sample into the platelet rich plasma and the blood cell sediment, it is realized that the preparation of the platelet rich plasma is completed on the disc during the detection process, without the need for pretreatment of the platelet rich plasma and without the need for arrangement of any additional device such as a centrifuge to prepare the platelet rich plasma in advance, thereby saving costs.

In an embodiment of the present application, after performance of the step S122, i.e. performance of disc centrifugation to separate the whole blood sample into the blood cell sediment and the platelet rich plasma, the platelet rich plasma moves to a plasma area, and the blood cell sediment moves to a sedimentation area.

Specifically, the disc in this embodiment is provided with the plasma area and the sedimentation area. After the whole blood sample is separated, the platelet rich plasma moves to the plasma area, and the blood cell sediment moves to the sedimentation area.

In this embodiment, when the step S122 is performed to centrifuge the disc and transfer the whole blood sample from the sample addition area to the plasma area of the disc, during centrifugation a rotation speed may be 2,000, an acceleration may be 3,000, a deceleration may be 3,000, a centrifugal time may be 120 seconds, and a rotation direction may be clockwise or counterclockwise.

After performance of the step S122, the step S200 is performed to centrifuge the disc to transfer the platelet rich plasma from the plasma area to the detection area of the disc, during centrifugation a rotation speed may be 2,000, an acceleration may be 2,000, a deceleration may be 500, a centrifugal time may be 10 seconds, and a rotation direction may be clockwise or counterclockwise.

In this embodiment, after the whole blood sample is separated into the blood cell sediment and the platelet rich plasma, the platelet rich plasma moves to the plasma area and the blood cell sediment moves to the sedimentation area, so that there is a specific area to hold the platelet rich plasma, which facilitates the subsequent migration of the platelet rich plasma to the detection area.

In an embodiment of the present application, the step S200 includes the following steps S211 to S212:

S211. the disc is centrifuged to transfer the platelet rich plasma to a quantitative area of the disc.

S212. the disc is centrifuged to transfer the platelet rich plasma in the quantitative area to a detection area of the disc.

Specifically, in this embodiment, a quantitative area is disposed on the disc, and the quantitative area is used for quantitatively migrating the platelet rich plasma to the detection area.

In this embodiment, by disposing the quantitative area, the platelet rich plasma is pre-quantified before migrating to the detection area.

In an embodiment of the present application, the step S200 includes the following steps S221 to S222:

S221. the disc is centrifuged to transfer the platelet rich plasma in the plasma area to the quantitative area of the disc.

S222. the disc is centrifuged to transfer the platelet rich plasma in the quantitative area to the detection area of the disc.

Specifically, in this embodiment, when the disc is centrifuged in the step S221, the platelet rich plasma in the plasma area is transferred to the quantitative area of the disc.

In this embodiment, since there are both a whole blood sample separation step and a pre-quantification step, the centrifugation parameters of each step are slightly different.

When the disc is centrifuged in the step S122 to transfer the platelet rich plasma from the sample addition area to the plasma area of the disc, during centrifugation a rotation speed may be 1,000, an acceleration may be 3,000, a deceleration may be 3,000, a centrifugal time may be 120 seconds, and a rotation direction may be clockwise or counterclockwise.

When the disc is centrifuged in the step S221 to transfer the platelet rich plasma from the plasma area to the quantitative area of the disc, during centrifugation a rotation speed may be 450, an acceleration may be 30,000, a deceleration may be 1,000, a centrifugal time may be 30 seconds, and a rotation direction may be clockwise or counterclockwise.

When the disc is centrifuged in the step S222 to transfer the platelet rich plasma from the quantitative area to the detection area of the disc, during centrifugation a rotation speed may be 1,000, an acceleration may be 1,000, a deceleration may be 1,000, a centrifugal time may be 10 seconds, and a rotation direction may be clockwise or counterclockwise.

In an embodiment of the present application, the centrifuging the disc includes:
driving the disc to rotate in a preset direction and for a preset duration.

Specifically, in the present application, in addition to centrifugally vibrating, a specific manner of centrifuging the disc may be to drive the disc to rotate in a preset direction and for a preset duration. The driving the disc to rotate in a preset direction and for a preset duration is driving the disc to rotate towards a preset direction and for a preset duration. The preset direction may be clockwise or counterclockwise. The preset duration may be preset before the detection.

In an embodiment of the present application, a plurality of detection areas, a plurality of sample addition areas, and a plurality of agent addition areas are disposed on the disc, each sample addition area having one detection area and one agent addition area corresponding to the sample addition area, or each sample addition area having a plurality of detection areas and a plurality of agent addition areas corresponding to the sample addition area.

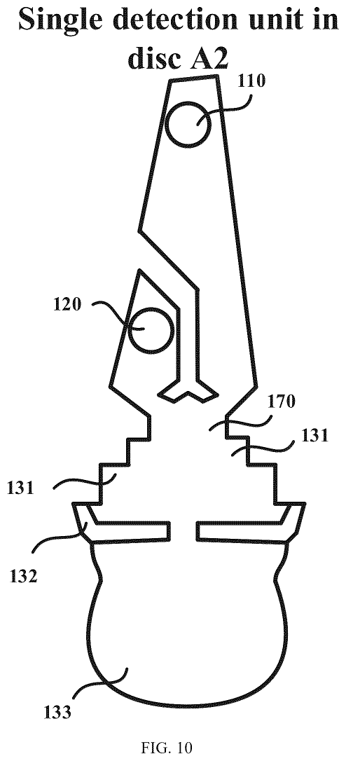

Specifically, in an embodiment, one sample addition area has one detection area and one agent addition area corresponding to the sample addition area, and the three correspond one to one. The quantitative areas disposed on the disc also correspond one-to-one to the detection areas, the agent addition areas, and the sample addition areas. Each detection area, each sample addition area, each inducing agent addition area and each quantification area constitute a detection unit. In this embodiment, a plurality of detection units are disposed on the disc, and the plurality of detection units are distributed in a ring shape on the disc; the positioning grooves surrounding the disc are arranged radially, as shown in FIG. 9; and the detection units are independent of each other, and two adjacent detection units will not affect liquid migration of each other.

In another embodiment, one sample addition area has a plurality of detection areas and a plurality of agent addition areas corresponding to the sample addition area. At this time, one whole blood sample (corresponding to the embodiment having a whole blood sample separation step and completing the preparation of the platelet rich plasma on the disc) or a piece of platelet rich plasma (corresponding to the embodiment in which the prepared platelet rich plasma is directly added on the disc) can be subjected to different detection items, thereby achieving diversified detection.

manner for introducing the platelet rich plasma into the addition area of the disc may be adding a sample through a sample injection needle. A single sample addition volume is within a numerical range of greater than or equal to 80 μL and less than or equal to 120 μL. Optionally, the single sample addition volume may be 108 μL.

A manner for introducing the inducing agent into the agent addition area of the disc may be adding a sample through a sample injection needle. A single sample addition volume is within a numerical range of greater than or equal to 5 μL and less than or equal to 20 μL. Optionally, the single injection volume may be 12 μL. When the liquid is replaced in the sample injection needle, an operation of washing the sample injection needle should be performed.

In this embodiment, by disposing a plurality of detection areas and a plurality of addition areas on the disc, and allowing each detection area to have one addition area corresponding to the detection area, the detection process can bear high throughput, can detect multiple samples at the same time, and has high detection efficiency. Moreover, it is also compatible with the detection work of multiple projects.

In an embodiment of the present application, the step S500 includes:

S510. the disc is driven to rotate until one detection area reaches a position opposite to the light source, so that the light source is projected onto the detection area.

S520. the disc is driven to stop rotating for a preset stopping time.

S530. the disc is driven to rotate again until the next detection area reaches the position opposite to the light source.

Specifically, this embodiment is a specific process of centrifugally vibrating, i.e., a process of detecting and sampling a suspension formed after mixing and reacting the platelet rich plasma and the inducing agent. The steps S510 to S520 need to be performed repeatedly. After the steps S510 to S520 are performed once, the sampling of one detection area is completed. Optionally, the time for performing the steps S510 to S520 lasts for M/N seconds, where N is the total number of the detection units, that is, the moving and sampling of all detection areas is completed within M seconds, and it takes M/N seconds for the moving and sampling of each detection area. The position opposite to the light source is the setting position of a light-through hole. The light source always emits light. When the disc rotates to the setting position of the light-through hole, the light emitted by the light source is irradiated on the detection area through the light-through hole, and then is irradiated on a light receiving tube in an optical assembly after transmitting through the suspension in the detection area. N may be 16. M may be 1.

The light is irradiated at the bottom of the detection area, and is emitted from the top of the detection area after transmission. The light intensity will weaken after passing through the suspension. The light rotates once every second, which means that the light is emitted for 16 times. Each time node has the light intensity of each detection area. As the reaction changes over 300 seconds, a reaction curve is generated. There are multiple reaction curves, with the horizontal axis being time and the vertical axis being light intensity.

In this step, during the centrifugally vibrating, a rotation speed may be 60, an acceleration may be 15,000, a total centrifugal time may be 300 seconds, and a rotation direction may be clockwise or counterclockwise.

S520. the disc is driven to stop rotating for a preset stopping time, which is actually the time for performing light transmission detection on the suspension formed after the platelet rich plasma and the inducing agent are mixed and reacted.

After the performance of the step S520 is completed once, the disc is driven to rotate again until the next detection area reaches the position opposite to the light source, where the rotation manner may be various. Optionally, one rotation manner is that directly rotating from one detection area to the next detection area, i.e., a step magnitude of 1 detection area in single time of movement. Optionally, another rotation manner is to first move by a step magnitude of 2 detection areas at a time, and then retreat by a step magnitude of one detection area. Different rotation plans can be formulated according to different detection projects.

In this embodiment, mixing uniformly while vibrating can be achieved in this way, and the amount of blood required to be added in a single time of stirring is greatly reduced compared to that of traditional external magnetic rod stirring. Moreover, this embodiment adopts face vibration sampling instead of vertical rotation sampling, which is a rare detecting and sampling manner on the market.

In an embodiment of the present application, when the step S500 is performed, that is, when the disc is centrifugally vibrated to allow the platelet rich plasma and the inducing agent to mix and react, the ratio of the volume of the suspension formed after the platelet rich plasma and the inducing agent in the detection area are mixed and reacted to the volume of the detection area is within a percentage range greater than or equal to 50% and less than or equal to 100%.

Specifically, the ratio of the volume of the suspension formed after the platelet rich plasma and the inducing agent in the detection area are mixed to the volume of the detection area may be 50%. The ratio of the volume of the suspension formed after the platelet rich plasma and the inducing agent in the detection area are mixed to the volume of the detection area may be 60%. The ratio of the volume of the suspension formed after the platelet rich plasma and the inducing agent in the detection area are mixed to the volume of the detection area may be 70%. The ratio of the volume of the suspension formed after the platelet rich plasma and the inducing agent in the detection area are mixed to the volume of the detection area may be 80%. The ratio of the volume of the suspension formed after the platelet rich plasma and the inducing agent in the detection area are mixed to the volume of the detection area may be 90%. The ratio of the volume of the suspension formed after the platelet rich plasma and the inducing agent in the detection area are mixed to the volume of the detection area may be 100%.

TABLE 1
detection results of ratios of the volumes of different
suspensions to the volume of the detection area

| | | | | | Average | | |
|---|---|---|---|---|---|---|---|
| | | Aggregation | Aggregation | Aggregation | value of | | |
| Sample | | Rate for test | Rate for test | Rate for test | aggregation | | Relative |
| No. | Ratio | 1 | 2 | 3 | rates | CV | deviation |
| Sample | 60% | 63.9% | 63.3% | 64.1% | 63.8% | 0.65% | 0.16% |
| 1 | 70% | 62.9% | 63.9% | 63.8% | 63.5% | 0.87% | −0.21% |
| | 80% | 62.7% | 62.9% | 63.8% | 63.1% | 0.93% | −0.81% |
| | 90% | 59.1% | 60.1% | 61.2% | 60.1% | 1.75% | −5.55% |
| | 100% | 55.5% | 57.7% | 50.3% | 54.5% | 6.97% | −14.4% |
| | Control | 62% | 65% | 64% | 64% | 2.40% | |
| Sample | 60% | 83.6% | 88.2% | 87.1% | 86.3% | 2.78% | −1.56% |
| 2 | 70% | 85.3% | 86.8% | 88.2% | 86.8% | 1.67% | −1.03% |
| | 80% | 85.1% | 87.3% | 85.3% | 85.9% | 1.42% | −2.02% |
| | 90% | 82.5% | 83.1% | 82.4% | 82.7% | 0.46% | −5.70% |
| | 100% | 71.4% | 71.2% | 77.0% | 74.2% | 3.77% | −15.36% |
| | Control | 89% | 88% | 86% | 87.7% | 1.74% | |
| Sample | 60% | 93.3% | 95.1% | 94.8% | 94.4% | 1.02% | 1.14% |
| 3 | 70% | 94.5% | 91.0% | 95.4% | 93.6% | 2.48% | 0.32% |
| | 80% | 89.5% | 95.8% | 93.1% | 92.8% | 3.41% | −0.57% |
| | 90% | 92.7% | 93.0% | 90.3% | 92.0% | 1.61% | −1.43% |
| | 100% | 86.3% | 81.2% | 89.6% | 85.7% | 4.94% | −8.18% |
| | Control | 94% | 91% | 95% | 93.3% | 2.23% | |
| Sample | 60% | 33.5% | 31.7% | 35.2% | 33.5% | 5.23% | 1.41% |
| 4 | 70% | 31.8% | 33.6% | 34.0% | 33.1% | 3.54% | 0.41% |
| | 80% | 31.9% | 33.3% | 30.5% | 31.9% | 4.39% | −3.33% |
| | 90% | 31.8% | 33.0% | 32.5% | 32.4% | 1.86% | −1.72% |
| | 100% | 29.8% | 26.3% | 24.0% | 26.7% | 10.94% | −19.09% |
| | Control | 34% | 33% | 32% | 33.0% | 3.03% | |

The aforementioned table, namely Table 1, is the results of the repeatability CV and relative deviation of the ratio of the volumes of 5 different suspensions to the volume of the detection area in 5 times of detection. The control group is a theoretical result.

The volume of the detection area is 130 μL, a volume of the suspension corresponding to the ratio of 60% is 78 μL, a volume of the suspension corresponding to the ratio of 70% is 91 μL, a volume of the suspension corresponding to the ratio of 80% is 104 μL, a volume of the suspension corresponding to the ratio of 90% is 117 μL, and a volume of the suspension corresponding to the ratio of 100% is 130 μL. 78/130-60%, 91/130=70%, 104/130=80%, 117/130=90%, and 130/130=100%, which respectively correspond to the ratios of the volumes of 5 different suspensions to the volume of the detection area.

According to Table 1, it can be seen that the relative deviations of the detection results (i.e., aggregation rates) of most of the 4 ratios from the aggregation rate of the control group are less than or equal to 15%, which is in line with expectations.

The CV value is a repeatability CV value. When the CV value is smaller, the precision is smaller, and the detection result is better or more excellent. The relative deviation is the relative deviation of the aggregation rate. When the relative deviation of the aggregation rate is smaller, the detection result is better or the more excellent. It is necessary to observe the relative deviation of the CV value and the aggregation rate at the same time. When the two are both smaller at the same time, it is better.

The repeatability CV value and relative deviation of aggregation rate for the ratio of 100% are the worst, the three repeatability CV values and relative deviations of aggregation rate for ratios of 60%, 70% and 80% are moderate, and the repeatability CV values and relative deviations of aggregation rate for the ratio of 90% are the optimum.

The result is optimum when the ratio of the volume of the suspension formed after the platelet rich plasma and the inducing agent in the detection area are mixed to the volume of the detection area is 90%. At this ratio, the uniformly mixing effect of the platelet rich plasma and the inducing agent is the best.

It should be noted that allowing the volume of the suspension to be 50-100% of a total volume that the detection hole can accommodate can achieve sufficient mixing. Table 1 only shows the data results of 5 ratios of 60%, 70%, 80%, 90% and 100%, and does not list the situations for all percentages.

In this embodiment, allowing the volume of the suspension to be 50-100% of a total volume that the detection hole can accommodate can achieve sufficient mixing.

### Example 1

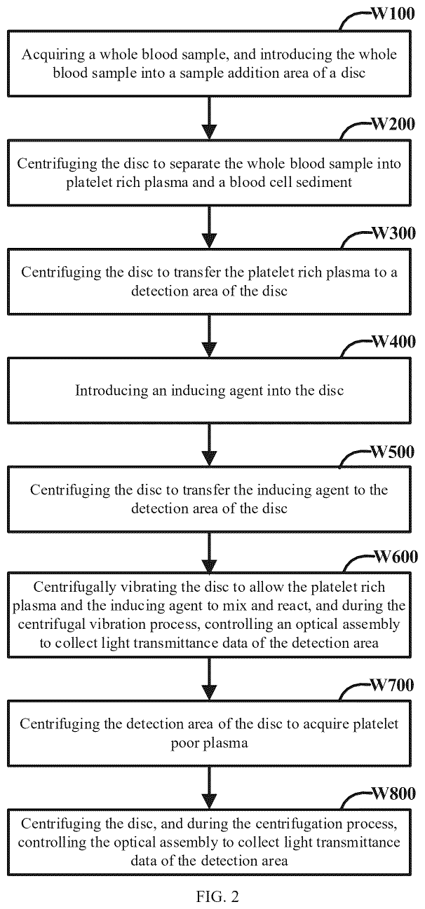

As shown in FIG. 2, the present application further provided a microfluidic fully-automatic platelet detection method, the method including the following steps W100 to W800:

W100. a whole blood sample was acquired, and introduced into a sample addition area of a disc.

W200. the disc was centrifuged to separate the whole blood sample into platelet rich plasma and a blood cell sediment.

W300. the disc was centrifuged to transfer the platelet rich plasma to a detection area of the disc.

W400. an inducing agent was introduced into the disc.

W500. the disc was centrifuged to transfer the inducing agent to the detection area of the disc.

W600. the disc was centrifugally vibrated to allow the platelet rich plasma and the inducing agent to mix and react, and during the centrifugal vibration process, an optical assembly was controlled to collect light transmittance data of the detection area.

W700. the detection area of the disc was centrifuged to acquire platelet poor plasma.

W800. the disc was centrifuged, and during the centrifugation process, the optical assembly was controlled to collect light transmittance data of the detection area.

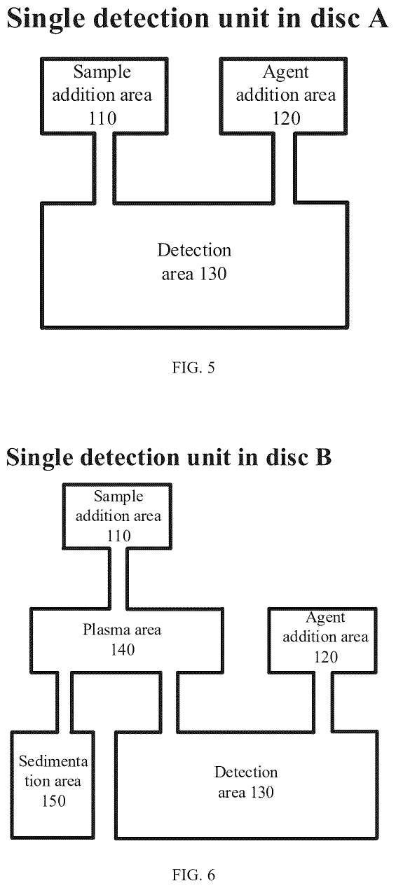

Specifically, this example was adapted to the disc B in FIG. 6 and the disc B1 in FIG. 11. The disc had a plasma area and a sedimentation area, but had no quantitative area.

### Example 2

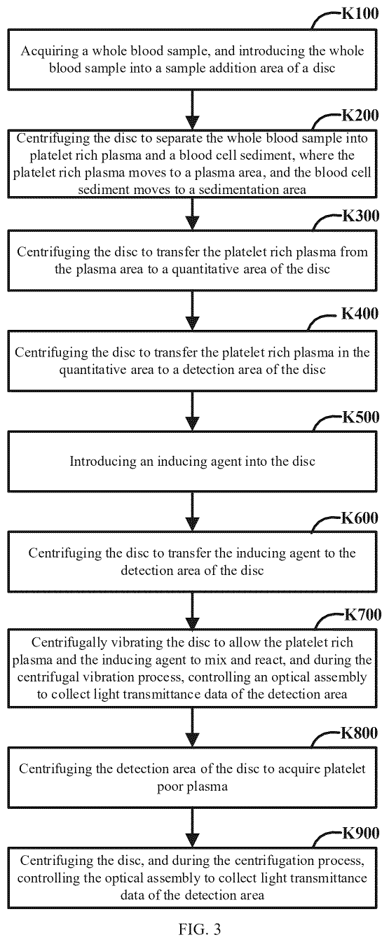

As shown in FIG. 3, the present application further provided a microfluidic fully-automatic platelet detection method, the method including the following steps K100 to K900:

K100. a whole blood sample was acquired and introduced into a sample addition area of a disc.

K200. the disc was centrifuged to separate the whole blood sample into platelet rich plasma and a blood cell sediment, where the platelet rich plasma moved to a plasma area, and the blood cell sediment moved to a sedimentation area.

K300. the disc was centrifuged to transfer the platelet rich plasma from the plasma area to a quantitative area of the disc.

K400. the disc was centrifuged to transfer the platelet rich plasma in the quantitative area to the detection area of the disc.

K500. an inducing agent was introduced into the disc.

K600. the disc was centrifuged to transfer the inducing agent to the detection area of the disc.

K700. the disc was centrifugally vibrated to allow the platelet rich plasma and the inducing agent to mix and react, and during the centrifugal vibration process, an optical assembly was controlled to collect light transmittance data of the detection area.

K800. the detection area of the disc was centrifuged to acquire platelet poor plasma.

K900. the disc was centrifuged, and during the centrifugation process, the optical assembly was controlled to collect light transmittance data of the detection area.

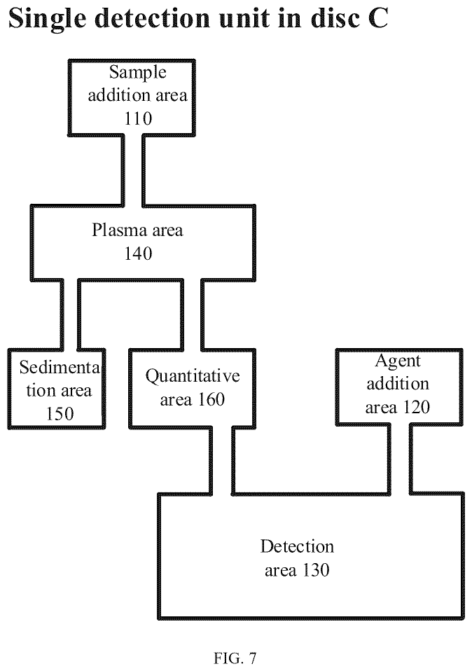

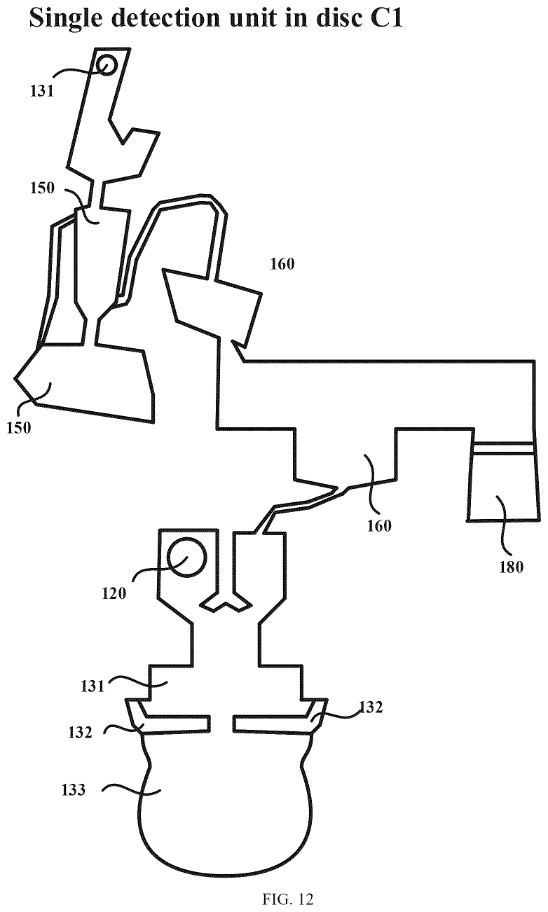

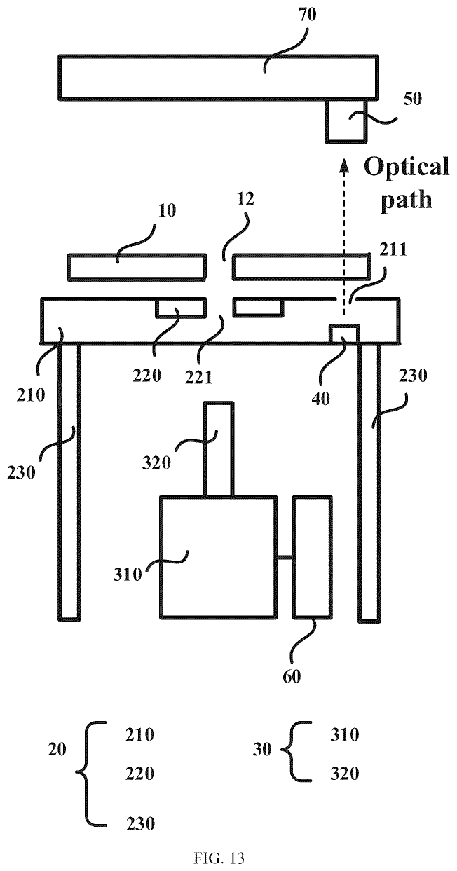

Specifically, this example was adapted to the disc C in FIG. 7 and the disc C1 in FIG. 12. The disc had not only a plasma area 140 and a sedimentation area 150, but also a quantitative area 160.

### Example 3

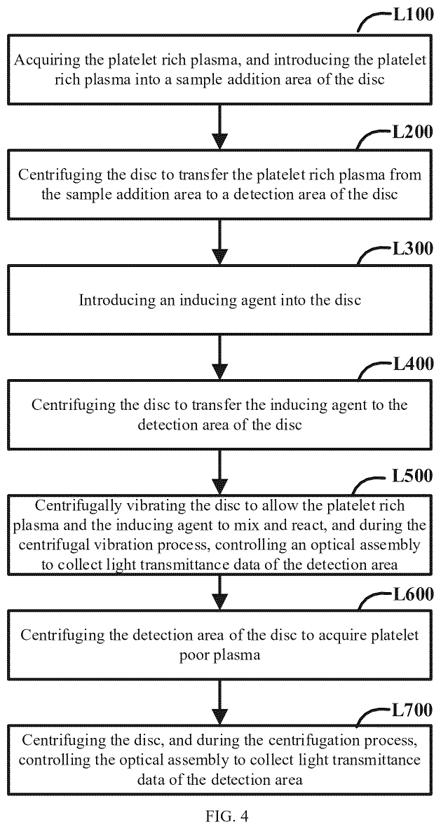

As shown in FIG. 4, the present application further provided a microfluidic fully-automatic platelet detection method, the method including the following steps L100 to L700:

L100. platelet rich plasma was acquired, and the platelet rich plasma was introduced into a sample addition area of the disc.

L200. the disc was centrifuged to transfer the platelet rich plasma from the sample addition area to a detection area of the disc.

L300. an inducing agent was introduced into the disc.

L400. the disc was centrifuged to transfer the inducing agent to the detection area of the disc.

L500. the disc was centrifugally vibrated to allow the platelet rich plasma and the inducing agent to mix and react, and during the centrifugal vibration process, an optical assembly was controlled to collect light transmittance data of the detection area.

L600. the detection area of the disc was centrifuged to acquire platelet poor plasma.

L700. the disc was centrifuged, and during the centrifugation process, the optical assembly was controlled to collect light transmittance data of the detection area.

Specifically, as shown in FIGS. 5, 8 and 9, this example was adapted to the disc A and the disc A1 in the figures. In this example, the platelet rich plasma was prepared outside the platelet analysis and homogenization system, and the prepared platelet rich plasma was directly added into the sample addition area 110 rather than on the disc. Since the platelet rich plasma was directly added into the sample addition area 110 and the preparation work was not completed on the disc, the disc had neither the plasma area 140 and the sedimentation area 150, nor the quantitative area 160.

The present application further provided a platelet analysis and homogenization system.

It should be noted that, for the sake of brevity, the designation of reference numerals for all component structures or areas was completed in the embodiments of the platelet analysis and homogenization system, and was not conducted in the embodiments of the microfluidic fully-automatic platelet detection method.

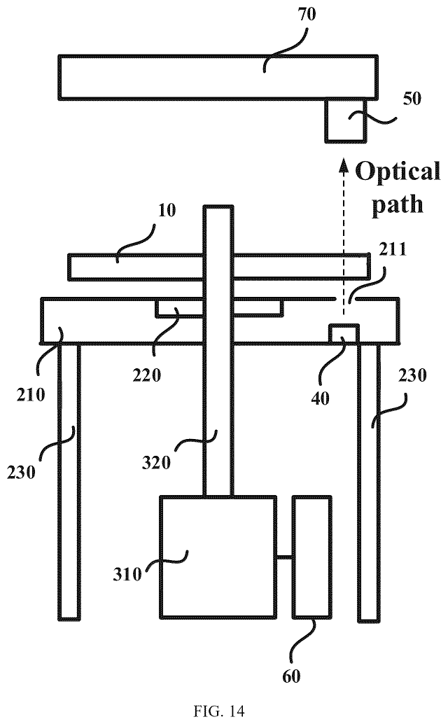

As shown in FIGS. 13 and 14, in an embodiment of the present application, the platelet analysis and homogenization system included a disc 10, a bearing device 20, a centrifugal device 30, a light source 40, an optical assembly 50, a first control device 60 and a second control device 70.

A first through hole 12 was opened at the center of the disc 10. The bearing device 20 included a tray 210, a rotating disc 220 and a supporting component 230. The rotating disc 220 was embedded in a groove on a top face of the tray 210. The tray 210 was provided with at least one light-through hole 211. A second through hole 221 was opened at the center of the rotating disc 220. The rotating disc 220 was further provided with a plurality of positioning holes, and the disc 10 was fixedly installed on the rotating disc 220 through the positioning holes. During installation, the first through hole 12 was aligned with the second through hole 221.

The centrifugal device 30 included a motor 310 and a motor shaft 320. The motor shaft 320 passed through the first through hole 12 and the second through hole 221, so that when the motor shaft 320 rotated, the rotating disc 220 and the disc 10 were driven to rotate in coordination. The light source 40 was disposed inside the tray 210. The light source 40 was located below the light-through hole 211. The optical assembly 50 was disposed above the rotating disc 220. The optical assembly 50 was disposed opposite to the light-through hole 211. The first control device 60 was electrically connected to the motor 310. The second control device 70 was electrically connected to the optical assembly 50.

Specifically, the first control device 60 might include a first PCB board. The second control device 70 might include a second PCB board. The optical assembly 50 might include a light receiving pipe, and the light receiving pipe might be welded on the second PCB board.

The platelet analysis and homogenization system might further include a single-chip microcomputer, which was electrically connected to the optical assembly 50 and was used for receiving the light transmittance data collected by the optical assembly 50.

Continually referring to FIGS. 13 and 14, in an embodiment of the present application, the disc 10 included a positioning groove 11 and a plurality of detection units 100 which were radially arranged around the positioning groove 11 with the positioning groove 11 as the circle center.

The detection unit 100 included a sample addition area 110, an agent addition area 120 and a detection area 130. The sample addition area 110 and the agent addition area 120 were both connected to the detection area 130. The detection area 130 included at least one barrier area 131, a partition board 132 and a detection hole 133. The barrier area 131 was disposed close to the sample addition area 110 and the agent addition area 120. The partition board 132 was disposed close to the barrier area 131. There were two partition boards 132, and the two partition boards 132 were disposed opposite to each other. The detection hole 133 was disposed close to the partition board 132.

Specifically, as shown in FIG. 9, the disc 10 included a positioning groove 11 and a plurality of detection units 100.

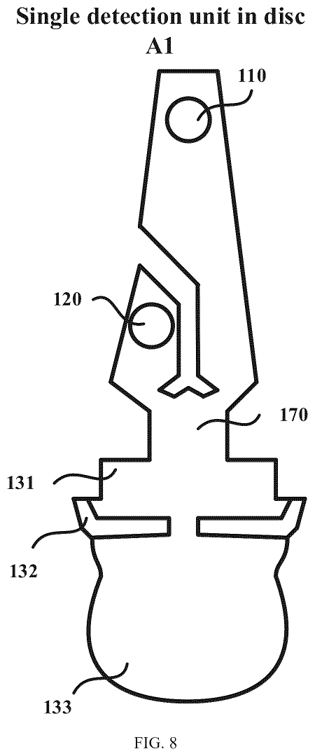

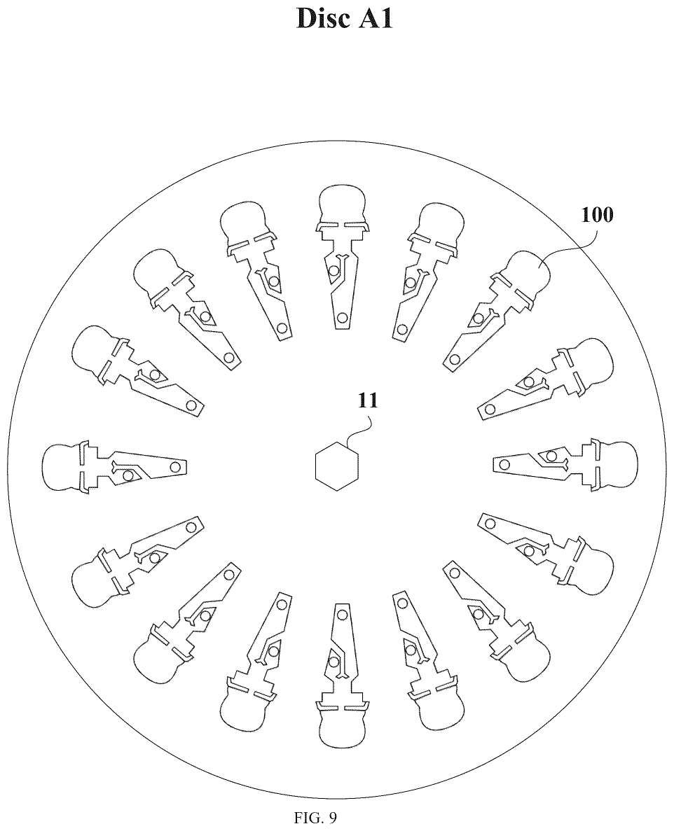

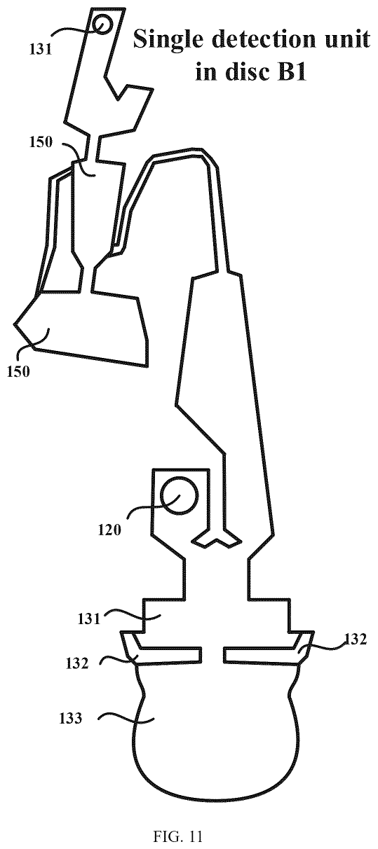

The function of the barrier area 131 was to prevent the suspension from flowing back when the suspension formed after mixing of the platelet rich plasma and the inducing agent at the position of the detection hole 133 was subjected to centrifugally vibrating and detection. In the embodiment as shown in FIG. 8, namely the disc A1 was shown to be a disc 10 having only one barrier area 131. In the embodiment as shown in FIG. 10, namely the disc A2 was shown to be a disc 10 having two barrier areas 131. The number of the barrier areas 131 was not limited, and the number of the barrier areas 131 was set to match that of different reaction systems to ensure that the suspension did not flow back into the channel 170 during vibration. Similarly, the disc B1 in FIG. 11 and the disc C1 in FIG. 12 were each provided with a barrier area 131, and their functions were the same.

The function of the partition board 132 was to slow down the flowing back of the suspension when the suspension formed after mixing of the platelet rich plasma and the inducing agent at the position of the detection hole 133 was subjected to centrifugally vibrating and detection. The disc A1 in FIG. 8, the disc A2 in FIG. 10, the disc B1 in FIG. 11, and the disc C2 in FIG. 12 were each provided with the partition board 132.

In an embodiment of the present application, the detection unit 100 further included a plasma area 140 and a sedimentation area 150. It was as shown in FIG. 11.

In an embodiment of the present application, the detection unit 100 further includes a quantification area 160 and a waste liquid pool 180. It was as shown in FIG. 12.

Specifically, when the disc was centrifuged in the step S221, the platelet rich plasma in the plasma area was transferred to the quantitative area 160 of the disc 10, and at the same time the excess platelet rich plasma entered the waste liquid pool 180.

The technical features of the aforementioned examples can be arbitrarily combined, and the execution order of the method steps is not limited. To simplify the description, we do not describe all possible combinations of the technical features in the aforementioned examples. However, as long as there is no contradiction in the combination of these technical features, it should be considered as the scope stated in this specification.

The examples described above are merely illustrative of several implementations of the present application, the description of them is more specific and detailed, but cannot be construed as limiting the scope of the present application accordingly. It should be noted that, several variations and modifications can be made by those of ordinary skills in the art, under the premise of not departing from the concept of the present application, and these variations and modifications all fall within the claimed scope of the present application. Therefore, the claimed scope of the present application shall be determined by the appended claims.

What is claimed is:

1. A microfluidic fully-automatic platelet detection method, the method comprising:
acquiring platelet rich plasma and introducing the platelet rich plasma into a disc;
centrifuging the disc to transfer the platelet rich plasma to a detection area of the disc;
introducing an inducing agent into the disc;
centrifuging the disc to transfer the inducing agent to the detection area of the disc;
centrifugally vibrating the disc to allow the platelet rich plasma and the inducing agent to mix and react, and during the centrifugal vibration process, controlling an optical assembly to collect light transmittance data of the detection area;
centrifuging the detection area of the disc to acquire platelet poor plasma; and
centrifuging the disc, and during the centrifugation process, controlling the optical assembly to collect light transmittance data of the detection area;
wherein, the centrifuging the disc comprises: driving the disc to rotate in a preset direction and for a preset duration;
the centrifugally vibrating the disc to allow the platelet rich plasma and the inducing agent to mix and react, and during the centrifugal vibration process, controlling an optical assembly to collect light transmittance data of the detection area, comprises:
driving the disc to rotate until one detection area reaches a position opposite to the light source, so that the light source is projected onto the detection area opposite to the light source;
driving the disc to stop rotating for a preset stopping time; and
driving the disc to rotate again until the next detection area reaches the position opposite to the light source.

2. The microfluidic fully-automatic platelet detection method according to claim 1, wherein the acquiring platelet rich plasma and introducing the platelet rich plasma into a disc comprises:
acquiring the platelet rich plasma, and introducing the platelet rich plasma into a sample addition area of the disc.

3. The microfluidic fully-automatic platelet detection method according to claim 1, wherein the acquiring platelet rich plasma and introducing the platelet rich plasma into a disc comprises:
acquiring a whole blood sample, and introducing the whole blood sample into a sample addition area of a disc; and
centrifuging the disc to separate the whole blood sample into platelet rich plasma and a blood cell sediment.

4. The microfluidic fully-automatic platelet detection method according to claim 3, wherein after the disc is centrifuged to separate the whole blood sample into a blood cell sediment and platelet rich plasma, the platelet rich plasma moves to a plasma area and the blood cell sediment moves to a sedimentation area.

5. The microfluidic fully-automatic platelet detection method according to claim 3, wherein the centrifuging the disc to transfer the platelet rich plasma to a detection area of the disc comprises:
centrifuging the disc to transfer the platelet rich plasma to a quantitative area of the disc; and
centrifuging the disc to transfer the platelet rich plasma in the quantitative area to a detection area of the disc.

6. The microfluidic fully-automatic platelet detection method according to claim 4, wherein the centrifuging the disc to transfer the platelet rich plasma to a detection area of the disc comprises:
centrifuging the disc to transfer the platelet rich plasma in the plasma area to the quantitative area of the disc; and
centrifuging the disc to transfer the platelet rich plasma in the quantitative area to a detection area of the disc.

7. The microfluidic fully-automatic platelet detection method according to claim 1, wherein a plurality of detection areas, a plurality of sample addition areas, and a plurality of agent addition areas are disposed on the disc, each sample addition area having one detection area and one agent addition area corresponding to the sample addition area, or each sample addition area having a plurality of detection areas and a plurality of agent addition areas corresponding to the sample addition area.

8. The microfluidic fully-automatic platelet detection method according to claim 7, wherein when the disc is centrifugally vibrated to allow the platelet rich plasma and the inducing agent to mix and react, the ratio of the volume of the suspension formed after the platelet rich plasma and the inducing agent in the detection area are mixed and reacted to the volume of the detection area is within a percentage range greater than or equal to 50% and less than or equal to 100%.

9. A microfluidic fully-automatic platelet detection method, the method comprising:
acquiring a whole blood sample, and introducing the whole blood sample into a sample addition area of a disc;
centrifuging the disc to separate the whole blood sample into platelet rich plasma and a blood cell sediment;
centrifuging the disc to transfer the platelet rich plasma to a detection area of the disc;
introducing an inducing agent into the disc;
centrifuging the disc to transfer the inducing agent to the detection area of the disc;
centrifugally vibrating the disc to allow the platelet rich plasma and the inducing agent to mix and react, and during the centrifugal vibration process, controlling an optical assembly to collect light transmittance data of the detection area;
centrifuging the detection area of the disc to acquire platelet poor plasma; and
centrifuging the disc, and during the centrifugation process, controlling the optical assembly to collect light transmittance data of the detection area; wherein the centrifuging the disc comprises:
driving the disc to rotate in a preset direction and for a preset duration;
the centrifugally vibrating the disc to allow the platelet rich plasma and the inducing agent to mix and react, and during the centrifugal vibration process, controlling an optical assembly to collect light transmittance data of the detection area, comprises:
driving the disc to rotate until one detection area reaches a position opposite to the light source, so that the light source is projected onto the detection area;
driving the disc to stop rotating for a preset stopping time; and
driving the disc to rotate again until the next detection area reaches the position opposite to the light source.

10. A microfluidic fully-automatic platelet detection method, the method comprising:
acquiring a whole blood sample, and introducing the whole blood sample into a sample addition area of a disc;
centrifuging the disc to separate the whole blood sample into platelet rich plasma and a blood cell sediment, where the platelet rich plasma moves to a plasma area, and the blood cell sediment moves to a sedimentation area;
centrifuging the disc to transfer the platelet rich plasma from the plasma area to a quantitative area of the disc;
centrifuging the disc to transfer the platelet rich plasma in the quantitative area to a detection area of the disc;
introducing an inducing agent into the disc;
centrifuging the disc to transfer the inducing agent to the detection area of the disc;
centrifugally vibrating the disc to allow the platelet rich plasma and the inducing agent to mix and react, and during the centrifugal vibration process, controlling an optical assembly to collect light transmittance data of the detection area;
centrifuging the detection area of the disc to acquire platelet poor plasma; and
centrifuging the disc, and during the centrifugation process, controlling the optical assembly to collect light transmittance data of the detection area;
wherein, the centrifuging the disc comprises:
driving the disc to rotate in a preset direction and for a preset duration;
the centrifugally vibrating the disc to allow the platelet rich plasma and the inducing agent to mix and react, and during the centrifugal vibration process, controlling an optical assembly to collect light transmittance data of the detection area, comprises:
driving the disc to rotate until one detection area reaches a position opposite to the light source, so that the light source is projected onto the detection area;
driving the disc to stop rotating for a preset stopping time; and
driving the disc to rotate again until the next detection area reaches the position opposite to the light source.

11. A platelet analysis and homogenization system for performing the microfluidic fully-automatic platelet detection method according to claim 1, comprising:
a disc with a first through hole opened at the center thereof;
a bearing device comprising a tray, a rotating disc and a supporting component, wherein the rotating disc is embedded in a groove on a top face of the tray, the tray is provided with at least one light-through hole, a second through hole is opened at the center of the rotating disc, the rotating disc is further provided with a plurality of positioning holes, the disc is fixedly installed on the rotating disc through the positioning holes, and during installation, the first through hole is aligned with the second through hole;
a centrifugal device comprising a motor and a motor shaft, wherein the motor shaft passes through the first through hole and the second through hole at the same time, so that when the motor shaft rotates, the rotating disc and the disc are driven to rotate in coordination;
a light source disposed inside the tray and located below the light-through hole;
an optical assembly disposed above the rotating disc and disposed opposite to the light-through hole;
a first control device electrically connected to the motor; and
a second control device electrically connected to the optical assembly.

12. The platelet analysis and homogenization system according to claim 11, wherein the disc comprises a positioning groove and a plurality of detection units arranged radially around the positioning groove with the positioning groove as the circle center;
the detection unit comprises:
a sample addition area;
an agent addition area; and
a detection area, wherein both the sample addition area and the agent addition area are connected to the detection area;
the detection area comprises:
at least one barrier area disposed close to the sample addition area and the agent addition area;
a partition board disposed close to the barrier area, wherein there are two partition boards, and the two partition boards are disposed opposite to each other, and
a detection hole disposed close to the partition board.
